(19) United States
(12) Patent Application Publication
(10) Pub. No.: US 2004/0034376 A1
(43) Pub. Date: Feb. 19, 2004

(54) Vasovasostomy stent

(76) Inventors: Mark Wildes, Bloomington, MN (US); Neil A. Stein, Plymouth, MN (US)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 10/223,109

(22) Filed: Aug. 15, 2002

(51) Int. Cl. A61B 17/08; A61F 2/06

(52) U.S. Cl. 606/153; 623/1.15

(57) ABSTRACT

A vasocasostomy stent of the present invention comprises an elongate tube including a wall defining an inner lumen and a plurality of apertures extending along a substantial length of the tube. A method of anastomosis for a vas deferens comprises inserting an apertured stent into free ends of a severed vas, slidingly advancing the free ends of the severed vas toward each other over the stent until the free ends are in abutment, and securing the free ends of the severed vas together over the stent.

### THE FIELD OF THE INVENTION

The present invention relates to a vascular stent and in particular to an anastomosis stent for reversing vasectomies.

### BACKGROUND OF THE INVENTION

With the right environment, bones can mend, nerves can regenerate, and skin will heal. Unfortunately, some things that are broken or damaged are extremely difficult to repair. This difficulty is particularly evident when the damage has been surgically caused in the first place.

For example, in a vasectomy, the vas deferens, which carries sperm from the testes to the urethra, is severed in two. The severed ends are then either tied off or left to freely dangle. In some cases, when the vas is separated, the cut includes removing a section of the vas to insure that the ends of vas cannot rejoin. A vasectomy is generally considered to be a permanent procedure.

However, like many elective procedures, vasectomy patients sometimes regret the procedure and seek to have a vasovasostomy (i.e., anastomisis of the vas), which attempts to reconnect the severed ends of the vas. With a reconnected vas deferens, the flow of sperm is restored to permit fertile ejaculation for conceiving a child.

Unfortunately, joining what has been separated is easier said than done. A wide catalog of vasectomy devices and vasectomy reversal devices has been presented for restoring a vas deferens. Many of these devices have been patented and include: biodegradable stents, such as those disclosed in U.S. Pat. No. 5,192,289, U.S. Pat. No. 5,425,739 and U.S. Pat. No. 4,674,506; removably insertable plugs, valves, stents, such as in U.S. Pat. No. 3,589,355, U.S. Pat. No. 4,682,592, U.S. Pat. No. 4,200,088, and U.S. Pat. No. 3,613,661; and various clamping and suturing devices, such as those disclosed in U.S. Pat. No. 4,245,638, U.S. Pat. No. 3,766,926, and U.S. Pat. No. 4,553,542. These patents, and many others directed to reversing a vasectomy, or directed to performing a reversible vasectomy, evidence the continuing and unfinished search for mastering the problem of restoring a severed vas deferens.

Several physiological factors unique to the vas deferens have acted in combination to frustrate prior attempts at reversing vasectomies. First, the opposed free ends of the severed vas frequently are of an unmatched diameter. In particular, the portion of the severed vas extending from the testes becomes dilated with spermatic buildup. On the other hand, the opposed free end of the severed vas retains its extremely small inner lumenal diameter in an undilated state. This inner lumenal mismatch between the free ends of the severed vas is quite difficult to work with in suturing together the free ends of the severed vas. Suturing the vas, with its small inner lumenal diameter, also creates risks such as stricture. A severed end of the vas also may be scarred and/or irregular shaped. If a section of the vas is removed during the vasectomy, rejoining the vas can be even more difficult due to the short length of available vas. This situation can necessitate a more radical procedure such as a vasoepididymostomy.

Accordingly, even with the wide variety of devices introduced over the last 40 years for solving the problem of performing vasectomies (and reversing them) the physiological factors surrounding vasectomies have prevented a satisfactory solution to reversing vasectomies.

### SUMMARY OF THE INVENTION

A vasocasostomy stent of the present invention comprises an elongate tube including a wall defining an inner lumen and a plurality of apertures extending along a substantial length of the tube. A method of anastomosis for a vas deferens comprises inserting an apertured stent into opposed free ends of a severed vas, slidingly advancing the opposed free ends of the severed vas toward each other over the stent until the free ends are in abutment, and securing the free ends of the severed vas together over the stent.

### BRIEF DESCRIPTION OF THE DRAWINGS

FIG. 1 is a perspective view of a vasovasostomy stent, according to an embodiment of the present invention.

FIG. 2 is a sectional view of FIG. 1 as taken along lines 2-2, according to an embodiment of the present invention.

FIG. 3 is a sectional view of FIG. 1 as taken along lines 3-3, according to an embodiment of the present invention.

FIG. 4 is a schematic view of preparing a vas for acceptance of a vasovasostomy stent of the present invention, according to an embodiment of the present invention.

FIG. 5 is a schematic view of insertion of a vasovasostomy stent into a vas, according to an embodiment of the present invention.

FIG. 6 is a schematic view of a vasovasostomy stent secured within a vas, according to an embodiment of the present invention.

FIG. 7 is a sectional view of FIG. 6 as taken along lines 7-7, according to an embodiment of the present invention.

### DESCRIPTION OF THE PREFERRED EMBODIMENTS

In the following detailed description of the preferred embodiments, reference is made to the accompanying drawings which form a part hereof, and in which is shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

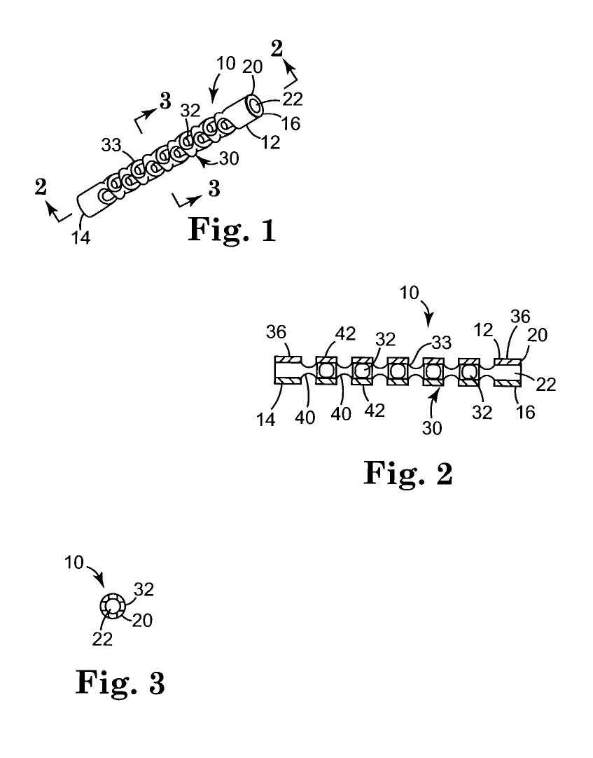

As shown in FIG. 1, vasovasostomy stent 10 comprises tube 12 with first end 14, second end 16, and wall 20 defining lumen 22. Wall 20 further comprises patterned body 30 defining a plurality of apertures 32 and undulating outer surface 33.

FIG. 2 is a sectional view of FIG. 1 as taken along lines 2-2. As shown in FIG. 2, stent 10 further comprises smooth surfaced end portions 36 and alternating recesses 40 and protrusions 42, which in combination with apertures 32, define undulating outer surface 33. Apertures 32 are distributed radially in a pattern about wall 20. Each aperture 32 defines an unobstructed pathway from the outside of stent 10 (e.g., outer surface 33) to the inside (e.g., inner lumen 22) of stent 10.

FIG. 3 is a sectional view of FIG. 1 as taken along lines 3-3. As shown in FIG. 3, wall 20 defines inner lumen 22 and apertures 32 of patterned body 30.

In one embodiment, stent 10 preferably has a length of about 0.10 inches, an outer diameter of about 0.01 inches (i.e., about 0.25 mm), and an inner diameter for lumen 22 of about 0.006 inches (i.e. about 0.15 mm). This length is sufficient to facilitate suturing the free ends of a severed vas, yet short enough to be less than an expected radius of curvature of the vas during normal physiologic use and functioning. The outer diameter of stent 10 is about two to three times the undilated, inner lumenal diameter of the vas, yielding an oversized vasvasostomy stent. Meanwhile, the inner lumenal diameter of inner lumen 22 of stent 10 is at least substantially the same as the inner lumenal diameter of vas (e.g., about 0.1 mm), and is preferably even one and one-half to two times larger (e.g., about 0.15 to 0.2 mm) than the inner lumenal diameter of vas, to ensure patentcy and prevent stricture at the suturing site, even after accounting for epithelial growth. Smooth surfaced end portions 36 preferably have a length of about 0.0015 inches while patterned body portion 30 (defining a plurality of apertures 32) has a length of about 0.077 inches. Apertures 32 preferably have a diameter of about 0.007 inches (i.e., about 0.18 mm) while wall 20 preferably has a thickness of about 0.002 inches. While apertures 32 are preferably generally circular shaped, apertures 32 optionally are formed from one or more different shapes such as oblong shapes, rectangular shapes, slots, etc. that define unobstructed passages through wall 20.

While the above dimensions identify one embodiment of the present invention, stent 10 optionally is made in different sizes, either smaller or larger, to the extent necessary to accommodate a differently sized vas deferens. In these cases, an inner diameter of stent 10 is sized to maintain a relationship of being substantially the same as an inner lumenal diameter of an undilated vas deferens, and even one and one-half to two times as larger than the inner lumenal diameter of the vas deferens. Likewise, the outer diameter of stent 10 is sized to maintain a relationship of being about two to three times larger than the inner lumenal diameter of an undilated vas deferens.

Stent 10 preferably is made from Nitonol, a well known surgical alloy, and alternatively can be made from other known surgical metallic alloys including stainless steel, Titanium, etc. Alternatively, stent 10 is made from semi-flexible, resilient non-metallic material with sufficient rigidity to maintain the patentcy of the vas. In each case, stent 10 preferably is made from a permanent, non-absorbable material.

Stent 10 is sized for insertion into opposed severed ends of a vas for maintaining the free ends of a severed vas in abutment with each other to facilitate suturing in order to reverse a vasectomy. Undulating surface 33 of stent 10 facilitates frictional engagement of vas against stent 10 during suturing of the free ends of the severed vas while apertures 32 of stent 10 facilitate epithelial tissue growth through stent 10 for vas to establish its own smooth wall lining that is enmeshed with lumen 22 of stent 10. In particular, the unobstructed passages formed by apertures 32 facilitate epithelial growth from outer surface 33 of stent, through apertures 32, and into and along inner lumen 22 of stent 10. This enmeshed combination of epithelial lining and stent 10 produce an inner lumenal diameter of the restored vas deferens that is more than sufficient to carry sperm, and which insures patentcy at the point of suture, largely due to the rigidity and large diameter of inner lumen 22 of stent 10. Finally, the outer diameter of stent 10 is sufficiently large to avoid the prior art sealing problem from mismatched diameters of free ends of a severed vas, since the outer diameter of stent 10 is larger than the sometimes dilated free vas end extending from the testes.

A method of the present invention for reversing a vasectomy with a vasovasostomy stent, according to an embodiment of the present invention, is shown in FIGS. 4-7. FIG. 4 schematically illustrates preparing two separated portions of a vas for reconnection with stent 10. Since an inner lumenal diameter of a vas is very small, this method first includes temporarily expanding this inner lumenal diameter of a vas with a dilator. As shown in FIG. 4, vas 50 includes two separated end portions 51 and 52, each having end surface 53, outer surface 54, inner lumen 56, and wall 58. Dilator 70 includes operative section 72 of an increasing diameter.

Using dilator 70, each severed end portion 51 and 52 of vas 50 is dilated one at a time. In particular, with severed end portions 51 and/or 52 temporarily secured, dilator 70 is separately introduced into each open end of severed end portions 51,52 of inner lumen 56 of vas 50 and advanced to expand the inner diameter of inner lumen 56 by stretching wall 58. Wall 58 of vas 50 is viscoelastic and therefore will the hold shape of the expanded inner diameter long enough to complete insertion and securing of stent 10 within vas 50. Dilator 70 preferably has an outer diameter that is two to three times the normal inner lumenal diameter of the vas, so that upon dilation of vas, stent 10 can be readily introduced and advanced into the vas. Any variety of dilators known in the art can be used.

As shown in FIG. 4, severed end portion 51 illustrates an undilated vas deferens prior to use of dilator 70 while severed end portion 52 illustrates a dilated vas deferens after use of dilator 70.

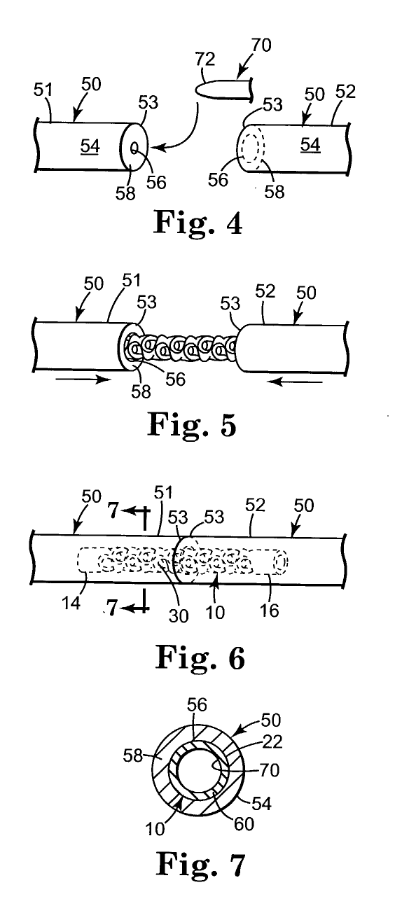

FIG. 5 illustrates stent 10 with its opposite ends 14,16 inserted in opposed severed end portions 51 and 52 of vas 50. With stent 10 positioned within severed end portions 51,52 of vas 50, each severed end portion 51,52 is then grasped and pulled together so that end surfaces 53 are in abutment over stent 10, as shown in FIG. 6. If desired various clamping and grasping devices known in the art are optionally used for handling the vas during advancement and suturing over stent 10.

FIG. 6 illustrates free ends portions 51 and 52 of severed vas 50 secured together over stent 10. This securing step is preferably performed using sutures, clips, lasers, and/or staples, all of which are known in the art and which are optionally woven into and through apertures 32 and lumen 22 of stent 10, as necessary.

FIG. 7 is a sectional view of stent 10 secured within restored vas 50. As shown, inner lumen 22 of stent 10 is relatively smooth due to epithelial growth from vas (extending through apertures 32 of patterned body 30 from outer surface 33 into inner lumen 22). This growth creates an enmeshed combination of epithelial lining 60 and of inner lumen 22 of stent 10 that defines new inner lumenal structure 70 that extends through stent 10.

The vasovasostomy stent of the present invention carries numerous advantageous features including patterned body with an undulating outer surface to facilitate suturing of the vas through frictional engagement of the stent against the vas inner lumen. The stent's oversized outer diameter insures sufficient patentcy of the restored vas while alleviating inner lumenal diameter mismatches of the severed vas ends by normalizing both inner lumenal diameters to a common-sized larger inner lumenal. Finally, the apertured wall of stent 10 enhances epithelial growth to form a smooth inner lumen wall lining within stent 10.

While specific embodiments have been illustrated and described, herein for purposes of description of the preferred embodiment, it will be appreciated by those of ordinary skill in the art that a wide variety of alternate and/or equivalent implementations may be substituted for the specific embodiments shown and described without departing from the scope of the present invention. Those with skill in the chemical, mechanical, electromechanical, electrical, and computer arts will readily appreciate that the present invention may be implemented in a very wide variety of embodiments. This application is intended to cover any adaptations or variations of the preferred embodiments discussed herein. Therefore, it is manifestly intended that this invention be limited only by the claims and the equivalents thereof.

What is claimed is:

1. A vasovasostomy stent comprising:
an elongate tube comprising:
a wall defining an inner lumen and a plurality of apertures extending along a substantial length of the tube.

2. The stent of claim 1 wherein the wall comprises opposite end portions defining a solid wall without apertures.

3. The stent of claim 1 wherein the wall comprises an undulating outer surface.

4. The stent of claim 3 wherein the undulating outer surface comprises a plurality of alternating recesses and protrusions.

5. The stent of claim 1 wherein each aperture defines an unobstructed path from an outer surface of the stent, through the wall, and into an inner lumen of the stent.

6. A vasovasostomy stent comprising:
an elongate tube comprising:
a wall defining an inner lumen and an undulating outer surface.

7. The stent of claim 5 wherein the wall comprises a plurality of apertures.

8. The stent of claim 1 wherein the apertures are arranged in radially distributed pattern.

9. The stent of claim 1 wherein the elongate tube is made from at least one of a generally rigid metallic material and a semi-flexible, resilient non-metallic material.

10. The stent of claim 1 wherein the elongate tube is made from a non-absorbable material.

11. The stent of claim 1 wherein the inner lumen defines an inner diameter that is about one to two times larger than an undilated vas deferens.

12. The stent of claim 1 wherein the outer surface defines an outer diameter that is about two to three times larger than undilated vas deferens.

13. A vasectomy reversal stent comprising:
an elongate tube comprising:
a wall defining an inner lumen and an outer surface;
a plurality of apertures extending along a substantial length of the tube, with each aperture extending through the wall from the outer surface to the inner lumen;
an undulating surface formed on the outer surface of the wall and extending along at least a portion of a length of the stent; and
a pair of opposite end portions defined by a solid wall portion free of apertures and free of an undulating surface, with the opposite end portions located at opposite ends of the plurality of apertures.

14. The stent of claim 13 wherein the elongate tube is made from a non-absorbable material that comprises at least one of a generally rigid metallic material and a semi-flexible, resilient non-metallic material; and
wherein the inner lumen defines an inner diameter that is about one to two times larger than an undilated vas deferens and the outer surface defines an outer diameter that is about two to three times larger than undilated vas deferens.

15. A vasovasostomy stent comprising:
an elongate tube including:
means for frictonally engaging an inner lumen of a vas; and
means for inducing epithelial growth of the vas through the stent.

16. A method of anastomosis for a severed vas deferens comprising:
inserting an apertured, non-absorbable stent into the free ends of a severed vas;
slidingly advancing the free ends of the severed vas together over the stent until the free ends are in abutment; and
securing the abutting free ends of the severed vas together over the stent.

17. The method of claim 16 wherein securing the free ends of the vas includes securing the free ends of the vas to the stent including at least one of suturing through apertures, suturing around the stent, and suturing through lumen of stent.

18. The method of claim 16 and further comprising:
prior to inserting the stent into free ends of vas, dilating lumen of severed ends of vas with a dilator

19. The method of claim 16 wherein dilating the vas includes:
removably inserting dilator into free ends of vas and advancing dilator to expand inner lumen of free end of vas.

20. The method of claim 16 wherein inserting an apertured stent comprises:
inducing epithethial growth from the inner lumen vas and the outer surface of the stent, through the apertures of the stent, and into and along the inner lumen of the stent.

21. The method of claim 16 wherein securing the free ends of the vas over the stent comprises:
maintaining a position of free end of vas over tube through frictional engagement of an undulating outer surface of the stent against an inner lumenal surface of the vas.

22. A method of establishing a patent vas in a vasovasostomy comprising:
disposing a stent within severed ends of vas wherein the stent has outer diameter greater than inner lumenal diameter of vas;
suturing ends of vas over stent;
maintaining stent within vas without removal of stent.
